(19) United States
(12) Patent Application Publication
(10) Pub. No.: US 2004/0116932 A1
(43) Pub. Date: Jun. 17, 2004

(54) Fixing screw

(76) Inventors: Keyvan Mazda, Paris (FR); Regis Le Couedic, Bordeaux (FR)

Correspondence Address: MASON & ASSOCIATES, PA, 17757 US HWY 19 N., SUITE 500, CLEARWATER, FL, 33764, US

(21) Appl. No.: 10/468,980

(22) PCT Filed: Feb. 22, 2002
(86) PCT No.: PCT/FR02/00658

(30) Foreign Application Priority Data
Feb. 22, 2001 (FR) 01/02372

(51) Int. Cl. A61B 17/58

(52) U.S. Cl. 606/73

(57) ABSTRACT

The invention relates to a fixing screw comprising a threaded longitudinal body (10) extending between a head (12) for driving it in rotation and a point-forming end (14) adapted to be inserted into a support. Said threaded longitudinal body (10) includes at least one break point (16) enabling said threaded longitudinal body (10) to be broken forcibly at said break point (16) in order to remove a portion (20) of said longitudinal body (10) extending from said break point (16) to said point-forming end (14), whereby said screw can be shortened prior to use.

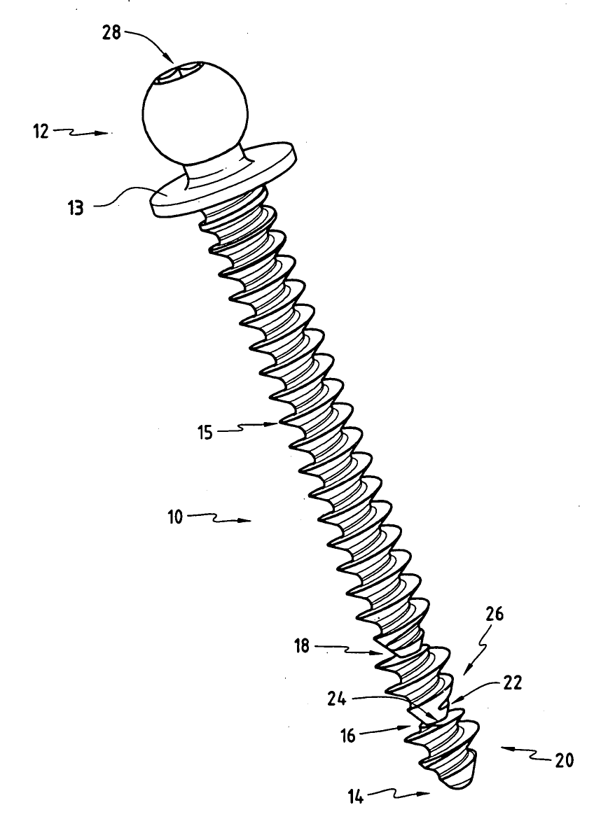

The present invention relates to a fixing screw comprising a threaded longitudinal body extending between a head for driving it in rotation and a point-forming end suitable for being inserted into a support.

One particular, although not exclusive, area of application envisaged is that of spine stabilizing systems which make it necessary to anchor fixing members into vertebrae by inserting into their pedicles longitudinal screws that extend into the vertebral body.

Spine stabilizing systems fix or guide movement of the vertebrae relative to each other in order to preserve the integrity of the spinal cord in patients with trauma or a degenerative pathology of the spine. To this end, fixing members are attached to the vertebrae by screws, known as pedicular screws, and the fixing members of superposed vertebrae are connected together by connecting rods to stiffen the spine.

The screws, each having a longitudinal body is threaded with a relatively large pitch from the head to the point-forming end, are inserted into the pedicle of the vertebra obliquely from the rear of the spine, until they reach the vertebral body. Two screws are generally inserted into the two pedicles of the vertebrae to attach two fixing members, one on each side of the axis of the spine, to obtain symmetrical stabilization.

However, the skeletal dimensions of persons fitted with a spine stabilizing system vary as do the cross-sections of the vertebrae. Thus the length of the pedicular screws used for attaching the fixing members must be chosen by the surgeon during the procedure, to which end the surgeon is provided with a selection of screws of different lengths.

Consideration has been given to making the longitudinal body of the screws easy to shorten to adapt their length to the intended use. One problem that then arises, and which the present invention aims to solve, is that of introducing into the support the new screw end obtained in this way.

To this end, in a first aspect, the present invention provides a screw whose threaded longitudinal body includes at least one break point to enable said threaded longitudinal body to be forcibly broken at said break point in order to remove a portion of said longitudinal body extending from said break point to said point-forming end and at least one frustoconical portion flared towards the head and extending from said break point towards the head to constitute a new point-forming end after said portion of said longitudinal body has been removed.

Thus one feature of the screw in accordance with the invention is its break points, which are situated at particular places along the threaded longitudinal body, and serve not only to enable the length of said body to be adapted to suit the dimensions of the vertebra into which it is to be screwed, but also to enable it to be inserted without difficulty. As a result of this, firstly, it is not necessary to provide a set of screws of different lengths, but instead to shorten a screw of standard length to the required length, and secondly, the frustoconical portion constitutes the new point-forming end, which facilitates screwing. Obviously, the standard length of the screws is chosen so that it is shorter than the maximum allowable length, and the position of the break point is chosen so that the length of the threaded body, once broken, is longer than the minimum acceptable length. Also, although the threaded longitudinal body has a break point to reduce the amount of force that must be applied in order to break it, a special mechanical device is provided to facilitate the operation. Ordinary cutters may be suitable.

In one particularly advantageous embodiment of the invention said break point is formed by a groove at the periphery of said threaded longitudinal body, whereby forcible displacement of said portion in a direction substantially perpendicular to said longitudinal body of the screw breaks said body in line with the bottom of said groove. Thus the break point is produced by a mechanical weakening of the threaded longitudinal body produced by a circular recess. Obviously, the groove that is formed in the longitudinal body is sufficiently strong to enable the screw to be screwed into a vertebra without breaking and sufficiently marked for the threaded longitudinal body to break easily.

In another particular embodiment of the invention, the wall of the groove nearer said head forms said frustoconical portion which is flared towards said head to constitute said new point-forming end after said threaded longitudinal body has been broken. Thus, by virtue of this feature, not only can the length of the threaded longitudinal body be reduced, but also the resulting end of the screw forms a point which facilitates forcible screwing into the vertebra.

It is particularly advantageous for said threaded longitudinal body to include a first break point and a second break point between said head and said first break point so that said threaded longitudinal body can be forcibly broken at said first break point to remove a first portion of said threaded longitudinal body extending from said first break point to said point-forming end or at said second break point to remove a second portion of said threaded longitudinal body extending from said second break point to said point-forming end.

As a result of this, the screw in accordance with the invention can have a length intermediate between a maximum length and minimum length. This feature enables better matching of the length of the screw to the dimensions of the vertebra into which it is to be inserted in order to obtain sufficient anchorage and to fasten the fixing member to the vertebra.

Said screw head preferably forms a ball in which there is formed a recess facing away from said threaded longitudinal body and adapted to receive a key to drive rotation of said head. The fixing member is therefore connected by appropriate means to the ball, around which it is partially articulated, prior to said member being clamped in order to immobilize it relative to the ball.

According to one advantageous feature the fixing screw in accordance with the invention is made of stainless steel. It is preferably made of titanium alloy, which is totally bio-compatible and offers good mechanical performance.

In another aspect, the present invention proposes a spine stabilizing system including at least one fixing screw in accordance with the invention adapted to be screwed into a pedicle of a vertebra.

Other features and advantages of the invention will emerge on reading the following description of particular embodiments of the invention, given by way of non-limiting example and with reference to the drawing, in which the sole FIGURE is a perspective view of one particular embodiment of a fixing screw of the invention.

The fixing screw shown in the sole FIGURE has a threaded longitudinal body 10 with a spherically symmetrical head 12 and it is terminated by a point-forming end 14. The longitudinal body 10 is threaded over the whole of its length and has a single thread of a special configuration and pitch suitable for the bone material into which the fixing screw is to be inserted.

The profile of the thread is an asymmetrical sawtooth such that the axial component of the action of contact with the material into which the screw is inserted is greater than the radial component. As a result of this, the threads 15 constitute effective abutments against the screw being pulled out along its longitudinal axis.

Although slightly rounded, the point-forming end 14 of the longitudinal body 10 is sufficiently sharp to enable said end 14 to be inserted into the bone material by pressing the end 14 with moderate force against said material and driving the screw in rotation.

An essential feature of the fixing screw of the invention is the provision of break points 16, 18 in the threaded longitudinal body 10; a first break point 16 is near the point-forming end 14, defining a portion 20 of said longitudinal body 10, and a second break point 18 is between the head 12 of the screw and the first break point 16.

The break points 16 and 18 are produced by reducing the cross-section of the threaded longitudinal body 10, to be more precise, and as shown in the single figure, by means of a circular groove at the periphery of the longitudinal body. The groove has two opposite walls inclined to each other and merging at the bottom of the groove. The wall of the groove 22 corresponding to the break point 16 which is at an angle of less than 90° to the axis of the fixing screw is flared towards the head 12 to form a truncated cone. In contrast, the opposite wall of the groove 22 is substantially perpendicular to the axis of the fixing screw. An identical configuration is adopted for the other break point 18.

The benefit of the break points 16 and 18 is obviously that they make it possible to break the longitudinal body 10 of the screw at one or the other of the break points 16 and 18 in order to match the length of the screw to the intended application. Apart from the dimensions of the skeleton, which determine the dimensions of the cross-section of the vertebrae and therefore the length required of a pedicular screw, fixing screws in accordance with the invention can be used in other applications in which the screw is inserted directly into the vertebral body from the front of the spine and for which the length of the screws is different. Thus, by making a fixing screw whose threaded longitudinal body is sufficiently long to cover all the possible maximum lengths that are encountered in various surgical procedures, and by providing at least two break points sufficiently far apart and sufficiently far from the point-forming end, it is possible to obtain a screw whose length can be adapted for use in all situations.

Also the invention is in no way limited to a longitudinal body in which only two break points are formed, but also covers a fixing screw whose longitudinal body includes three grooves for more appropriate matching of the length of the fixing screw.

The break points 16, 18 formed by the grooves enable the threaded longitudinal body 10 to be shortened to an appropriate length at the time of use. To this end a special device or standard cutters are used to move the portion 20, for example, in a direction substantially perpendicular to the axis of the fixing screw, while the screw is being held in a fixed position, so as to break the longitudinal body 10 opposite the break point 16. If cutters are used, their jaws can cut the longitudinal body more easily at a break point, where there is less material.

Also, because the flared shape of the wall of the groove 22 corresponding to the break point 16 forms a truncated cone, and because the break is substantially at the bottom of the groove, when the portion 20 is removed, where the two walls meet, this exposes a new point-forming end 24. This new point-forming end requires no particular machining and enables the screw to be inserted into the bone material.

Obviously, if the length of the longitudinal body 10 is to be reduced further, a procedure identical to that described above is carried out at the break point 18 between the head 12 and the break point 16, to remove the portion 26 of the longitudinal body.

Fixing screws in accordance with the invention are made of titanium alloy, stainless steel, or any other material having the same properties, and it goes without saying that when they still incorporate the grooves, because they have not been cut, they retain their mechanical strength not only to provide a secure fixing but also to withstand forcible insertion into the bone material.

The invention also provides a spine stabilizing system, not shown, including at least one screw whose threaded longitudinal body includes at least one break point to enable said threaded longitudinal body to be forcibly broken at said break point in order to remove a portion of said longitudinal body extending from said break point to said point-forming end. According to an advantageous feature of the system, the head of the screw forms a ball around which a fixing device can be articulated and on which it can be immobilized.

The head advantageously has a central recess 28 facing away from the threaded longitudinal body 10 and adapted to receive a key for driving rotation of the screw to insert it into the bone material.

1. A fixing screw comprising a threaded longitudinal body (10) extending between a head (12) adapted to be driven in rotation and a point-forming end (14) suitable for being inserted into a support, which screw is characterized in that said threaded longitudinal body (10) includes:
at least one break point (16) to enable said threaded longitudinal body (10) to be forcibly broken at said break point (16) in order to remove a portion (20) of said longitudinal body (10) extending from said break point (16) to said point-forming end (14), and
at least one frustoconical portion flared towards the head (12) and extending from said break point towards the head (12) to constitute a new point-forming end after said portion (20) of said longitudinal body (10) has been removed.

2. A fixing screw according to claim 1, characterized in that said break point (16) is formed by a groove at the periphery of said threaded longitudinal body (10), whereby forcible displacement of said portion (20) in a direction substantially perpendicular to said longitudinal body (10) of the screw breaks said body (10) in line with the bottom of said groove.

3. A fixing screw according to claim 2, characterized in that the wall of the groove (22) nearer said head (12) forms said frustoconical portion which is flared towards said head (12) to constitute said new point-forming end (24) after said threaded longitudinal body (10) has been broken.

4. A fixing screw according to any one of claims 1 to 3, characterized in that said threaded longitudinal body includes a first break point (16) and a second break point (18) between said head (12) and said first break point (16) so that said threaded longitudinal body (10) can be forcibly broken at said first break point (16) to remove a first portion (20). of said threaded longitudinal body (10) extending from said first break point (16) to said point-forming end (14) or at said second break point (18) to remove a second portion (26) of said threaded longitudinal body (10) extending from said second break point (18) to said point-forming end (14).

5. A fixing screw according to any one of claims 1 to 4, characterized in that said screw head (12) forms a ball in which there is formed a recess (28) facing away from said threaded longitudinal body (10), said recess (28) being adapted to receive a key to drive rotation of said head (12).

6. A fixing screw according to any one of claims 1 to 5, characterized in that it is made of stainless steel.

7. A fixing screw according to any one of claims 1 to 6, characterized in that it is made of titanium alloy.

8. A spine stabilizing system, characterized in that it includes at least one fixing screw according to any one of claims 1 to 7 adapted to be screwed into a pedicle of a vertebra.
